(19) United States
(12) Patent Application Publication
(10) Pub. No.: US 2010/0298739 A1
(43) Pub. Date: Nov. 25, 2010

(54) Multifunctional Medical Access Device

(75) Inventors: Gregory A. Steube, St. Charles, MO (US); Todd M. Chelak, Westborough, MA (US)

Correspondence Address: TYCO HEALTHCARE GROUP LP, 15 HAMPSHIRE STREET, MANSFIELD, MA, 02048, US

(73) Assignee: TYCO HEALTHCARE GROUP LP, Mansfield, MA (US)

(21) Appl. No.: 12/848,575

(22) Filed: Aug. 2, 2010

Related U.S. Application Data
(62) Division of application No. 12/109,481, filed on Apr. 25, 2008.
(60) Provisional application No. 60/926,333, filed on Apr. 26, 2007.

(51) Int. Cl. A61M 5/32; A61B 5/153

(52) U.S. Cl. 600/576; 604/198; 604/411

(57) ABSTRACT

A medical access device is provided including a syringe needle and a safety shield supportable on the syringe needle. The safety shield includes a pair of spaced legs each having a distal segment and a proximal segment, each of the proximal segments having a proximal end fixed with respect to the syringe needle and a distal end hingedly attached to the distal segment at a hinge member, each proximal segment including a camming surface. The safety shield also includes a nose member connected to a distal end of each distal segment of the pair of spaced legs. The safety shield also includes a trigger being movable to move the camming member into engagement with the camming surfaces of the proximal segments to effect movement of the legs between a first position and a second position.

### CROSS-REFERENCE TO RELATED APPLICATION

The present application is a Divisional Application claiming the benefit of and priority to U.S. application Ser. No. 12/109,481, filed on Apr. 25, 2008, which claims the benefit of and priority to U.S. Provisional Application Ser. No. 60/926,333, filed on Apr. 26, 2007, the entire content of each of which is incorporated herein by reference for all purposes.

### BACKGROUND

1. Technical Field

The present disclosure relates to medical access devices and, more particularly, to medical access devices adapted for both needle access, for blood collection and the like, and for blunt tip access, for penetrating membranes or septums as commonly used in medication vials, intravenous bags, access ports and the like, and adapted to include a safety shield apparatus.

2. Description of Related Art

Safety shields for shielding needles of medical devices are well known in the art. Safety shields minimize the risks associated inadvertent needle stick injuries which subject doctors, nurses and medical personnel to exposure to HIV, hepatitis and other serious blood-borne pathogens.

It is known to incorporate a safety shield into the body of a medical needle. More specifically, it is known to form a safety shield apparatus integrally with a medical needle device, e.g., a blood collection device or a syringe needle. It is also known to provide a hub on the safety shield apparatus which includes a luer fitting to selectively secure the safety shield apparatus to a medical needle device. The hub can be formed integrally with or separately from the safety shield apparatus.

In use, following use of the medical needle to collect blood or to withdraw medication from a vial, the safety shield apparatus is actuated to shield the distal tip of the needle in order to properly and safely dispose of the needle or to disconnect the medical needle from the blood collector vial or from the syringe barrel of the syringe needle. With the medical needle removed, in certain applications, a blunt tip cannula may be attached to the syringe barrel in order to penetrate membranes or septums, without damaging the membranes or septums, to inject the collected blood or medication into an intravenous bag, an access port, a vial/test tube or the like.

Accordingly, a need exists in the art of medical devices for an inexpensive, simple device capable of functioning as a medical needle syringe and a blunt tip cannula including a multi-functional safety shield apparatus.

### SUMMARY

The present disclosure relates to medical access devices adapted for both needle access, for blood collection and the like, and for blunt tip access, for penetrating membranes or septums as commonly used in medication vials, intravenous bags, access ports and the like, and adapted to include a safety shield apparatus.

According to an aspect of the present disclosure, a medical access device is provided including a syringe needle having a proximal end and a distal end configured for percutaneous use and a deployable safety shield supported on the syringe needle. The safety shield includes a proximal segment having a proximal end and a distal end; a distal segment having a proximal end and a distal end, wherein the proximal end of the distal segment is connected to the distal end of the proximal segment; and a nose member having a proximal end and a distal end, wherein the proximal end of the nose member is connected to the distal end of the distal segment, wherein the nose member is translatably disposed on the syringe needle. The safety shield is movable between a first position wherein the nose member is retracted from the distal end of the syringe needle to expose the distal end of the syringe needle and a second position wherein the nose member is extended beyond the distal end of the syringe needle to shield the distal end of the syringe needle.

The medical access device may further comprise a needle hub supporting the proximal end of the syringe needle, wherein the needle hub is configured for selective connection with a complementary feature of a fluid source. The proximal segment and the distal segment of the safety shield may be pivotally connected to one another. The distal segment and the nose member of the safety shield may be pivotally connected to one another. The nose member of the safety shield may surround the syringe needle. The distal end of the nose member may be tapered. The distal end of the nose member may define a blunt tip.

A fluid-tight seal may be provided between the nose member of the safety shield and the syringe needle. The medical access device may further comprise a sealing member between the nose member of the safety shield and the syringe needle.

The safety shield may include a locking feature for maintaining the safety shield in the second position.

According to another aspect of the present disclosure, a medical access device is provided and includes a syringe needle having a proximal end and a distal end configured for percutaneous use and a deployable safety shield supportable on the syringe needle. The safety shield includes a body portion configured to selectively receive at least a portion of the syringe needle. The body portion has a distal end defining a nose member. The body portion is movable between an uncovered position where the distal end of the syringe needle is exposed and a covered position wherein the distal end of the syringe needle is shielded and the nose member extends beyond the distal end of the syringe needle to establish a blunt penetration tip for the medical access device, wherein when the body portion is in the covered position the nose member defines a passage into the syringe needle.

The safety shield may include a sealing member supported on the body portion, such that the sealing member is penetratable by the syringe needle when the body portion is in the covered position. The body portion may be axially movable with respect to the syringe needle. In use, when the body portion is in the covered position, the body portion may be axially displaced relative to the syringe needle by an amount sufficient for the distal end of the syringe needle to extend completely through the sealing member.

The distal end of the body portion may have a length sufficient to cover the distal end of the syringe needle when the body portion has been moved to a position where the distal end of the syringe needle extends distally beyond the sealing member.

The distal end of the body portion may be tapered. The body portion may include retaining members for inhibiting uncovering of the syringe needle when the body portion is in the covered position.

The medical access device may further include a needle hub configured to support the proximal end of the syringe needle and having the safety shield connected thereto. The body portion may be pivotably connected to the needle hub. The body portion may be axially movable with respect to the needle hub.

The needle hub may include a locking feature configured to maintain the body portion in a retracted position relative to the syringe needle.

According to a further aspect of the present disclosure, a medical access device is provided, including a syringe needle having a proximal end and a distal end configured for percutaneous use; and a safety shield supportable on the syringe needle. The safety shield includes a pair of spaced legs each having a distal segment and a proximal segment, each of the proximal segments having a proximal end fixed with respect to the syringe needle and a distal end hingedly attached to the distal segment at a hinge member, each proximal segment including a camming surface; a nose member connected to a distal end of each distal segment of the pair of spaced legs, wherein the nose member is translatably disposed on the syringe needle, wherein the nose member defines a blunt tip; and a trigger having a camming member and being movable to move the camming member into engagement with the camming surfaces of the proximal segments to effect movement of the legs between a first position in which the nose member and the distal ends of the distal segments shield at least a distal end of the syringe needle and a second position in which the distal end of the syringe needle is shielded by the nose member and capable of being at least partially exposed from a distal end of the nose member when the nose member is moved in a proximal direction relative to the syringe needle.

In use, when in the first position, the distal and proximal segments are substantially linearly aligned with the hinge member of each leg positioned adjacent the needle such that when a force, acting in a substantially linear proximal direction, is applied to a distal end of the nose member of the safety shield, the legs are retained in the first position.

The camming member of the trigger and the camming surfaces of the proximal segments may be positioned and configured such that movement of the camming member of the trigger into engagement with the camming surfaces of the proximal segments splays the hinge member of each of the legs outwardly to retract the nose member in a proximal direction relative to the syringe needle.

Each of the legs may define a channel dimensioned to receive the syringe needle.

The medical access device may further comprise a needle hub configured for selective connection with a complementary feature of a fluid source, wherein the trigger is pivotally secured to the needle hub. The trigger may include an attachment end configured for selective pivotable attachment to the needle hub.

The medical access device may further comprise a biasing member configured to urge the legs to the first position.

The camming member of the trigger may include a pair of spaced cam portions, wherein each of the cam portions is configured to engage one of the camming surfaces of the proximal segments. The proximal end of each camming portion may define an engaging surface for engaging a shoulder on one of the proximal segments. The distal end of at least one of the camming portions may extend below a bottom-most surface of the safety shield.

The camming member of the trigger may include at least one engaging surface and the camming surfaces of the proximal segments include at least one shoulder, the at least one engaging surface being movable into engagement with the at least one shoulder to retain the trigger in engagement with the proximal segments and retain the legs in the second position.

According to a further aspect of the present disclosure, a medical access device is provided, including a syringe needle having a proximal end and a distal end, the syringe needle slidably extending through at least a body portion of a nose member defining a blunt tip and defining a longitudinal axis therethrough; a pair of spaced legs each having a distal segment and a proximal segment, the distal segments cooperating with the proximal segments via hinge members, the legs adapted to act as a safety shield pivotally connected to the syringe needle via at least one retention member; and a trigger mechanism supported on a needle hub or the retention member, the trigger mechanism having a lever and a camming member, the trigger mechanism configured to move the camming member into engagement with camming surfaces of the proximal segments of the legs to effect movement of the legs between a first position and a second position, the movement of the legs being perpendicular to the longitudinal axis defined by the syringe needle.

### BRIEF DESCRIPTION OF THE DRAWINGS

Various embodiments of the presently disclosed medical access device are disclosed herein with reference to the drawings, wherein:

FIG. 1 is a perspective view of an embodiment of the presently disclosed medical access device illustrating a multi-functional safety shield apparatus thereof in a first position, and illustrating its selective connection to an exemplary needle syringe;

FIG. 2 is an exploded perspective view of the medical access device of FIG. 1;

FIG. 3 is a front, elevational view of the medical access device of FIGS. 1 and 2;

FIG. 4 is a longitudinal, cross-sectional view of the medical access device of FIGS. 1-3, as taken through 4-4 of FIG. 3;

FIG. 5 is an enlarged view of the indicated area of detail of FIG. 4;

FIG. 6 is a front, elevational view of the medical access device of FIGS. 1-5, illustrating the multi-functional safety shield apparatus in a partially deployed position;

FIG. 7 is a longitudinal, cross-sectional view of the medical access device of FIG. 6, as taken through 7-7 of FIG. 6;

FIG. 8 is a perspective view of the medical access device of FIGS. 1-7, illustrating the multi-functional safety shield apparatus in a fully deployed position;

FIG. 9 is a top, plan view of a medical access device according to another embodiment of the present disclosure, shown in a first position;

FIG. 10 is a longitudinal, cross-sectional view of the medical access device of FIG. 9 as taken through 10-10 of FIG. 9;

FIG. 11 is a top, plan view of the medical access device of FIGS. 9 and 10, shown in a second position;

FIG. 12 is a longitudinal, cross-sectional view of the medical access device of FIG. 11 as taken through 12-12 of FIG. 11;

FIG. 13 is a schematic illustration of a medical access device according to yet another embodiment of the present disclosure, shown in a first condition;

FIG. 14 is a schematic illustration of the medical access device of FIG. 13, shown in a second condition; and

FIG. 15 is a schematic illustration of the medical access device of FIGS. 13 and 14, shown in a third condition.

### DETAILED DESCRIPTION OF THE EMBODIMENTS

Embodiments of the presently disclosed medical access device and multi-functional safety shield will now be described in detail with reference to the drawings wherein like reference numerals designate identical or corresponding elements in each of the several views.

In the discussion that follows, the term “proximal” refers to a portion of a structure that is closer to a clinician, and the term “distal” refers to a portion that is further from the clinician. As used herein, the term “subject” refers to a patient that receives infusions or has blood and/or fluid collected therefrom using the medical access device. According to the present disclosure, the term “clinician” refers to an individual administering an infusion, performing fluid collection, installing or removing a needle cannula from a safety apparatus and may include support personnel or any other person contemplated of using the medical access device.

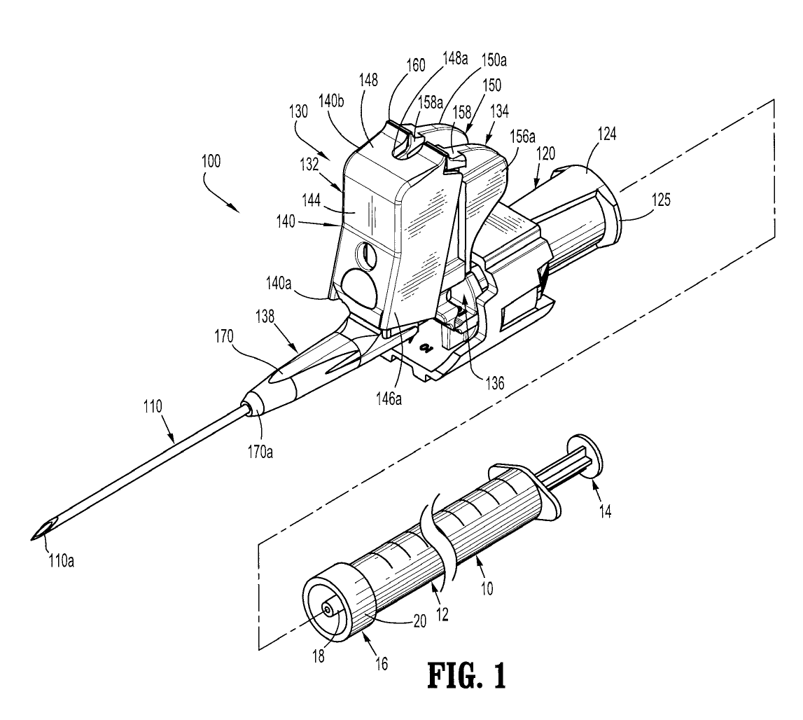

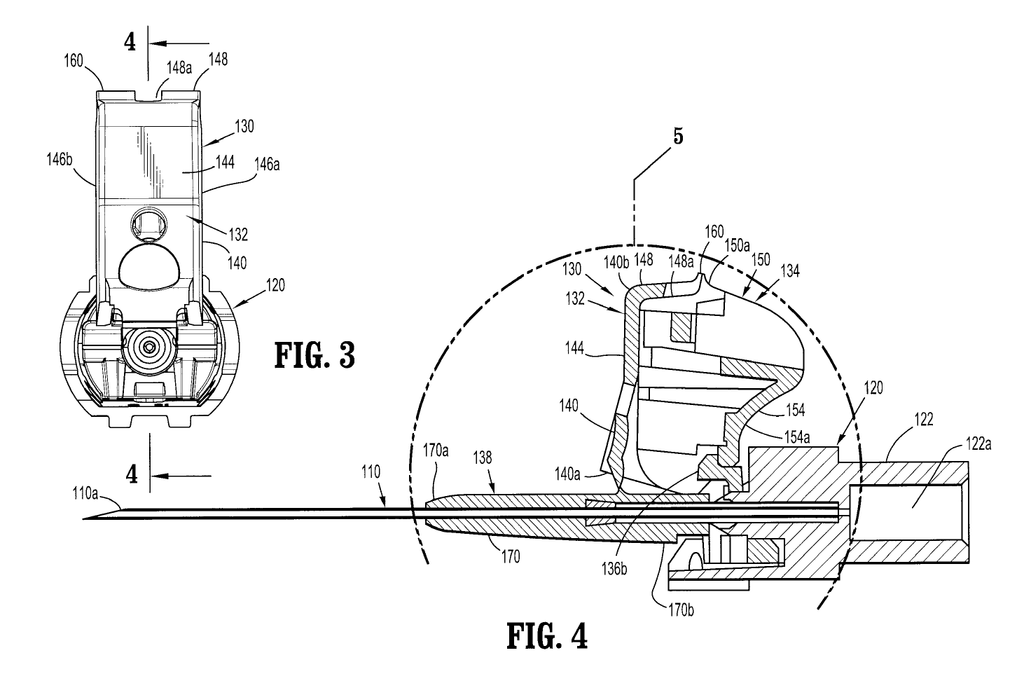

Referring now to the various figures of the drawings, there is shown in FIGS. 1-8, a medical access device 100 according to an embodiment of the present disclosure. As seen in FIG. 1, medical access device 100 may be selectively, fluidly connected to a fluid receptacle 10, in the form of a syringe or the like. Syringe 10 may include a syringe barrel 12 and a plunger 14 slidably disposed within syringe barrel 12. Syringe barrel 12 includes a distal end 16 having an elongate barrel tip 18 and a collar 20 extending distally therefrom.

As seen in FIGS. 1-8, medical access device 100 includes a syringe needle 110 supported on a needle hub 120, and a multi-functional safety shield 130 operatively mounted on needle hub 120 and slidably positioned about syringe needle 110. A distal end 110a of syringe needle 110 is tapered to enable tissue penetration and the like, a proximal end 110b of syringe needle 110 is fluidly connected to or supported within needle hub 120.

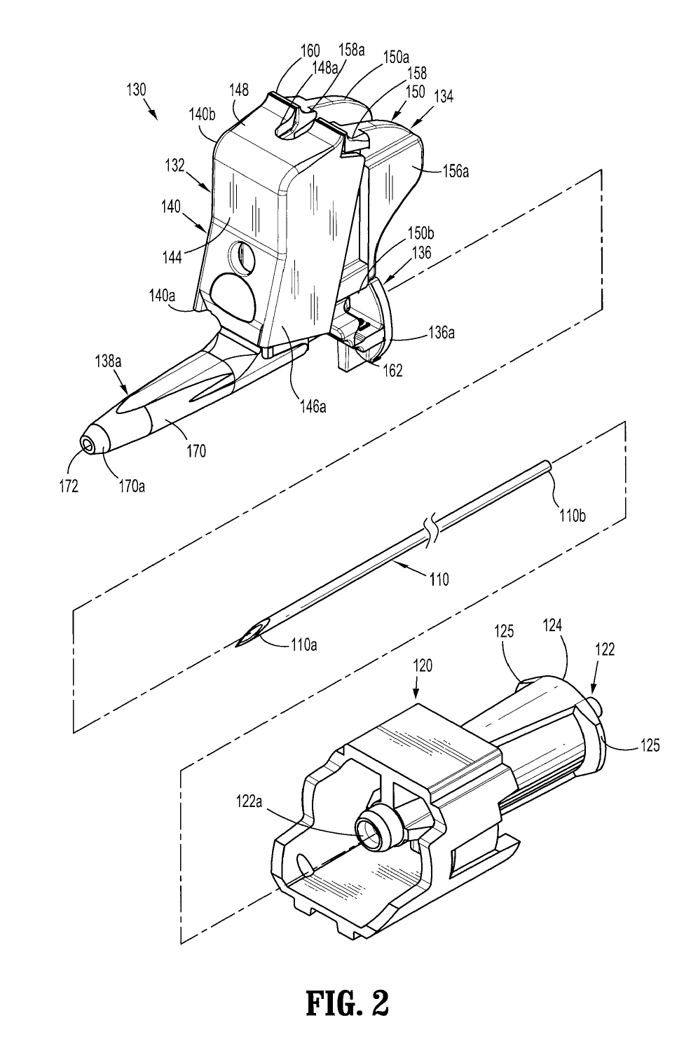

As seen in FIGS. 1 and 2, needle hub 120 includes a Luer-type connector having a needle support 122 defining a lumen 122a therethrough for support of syringe needle 110 therein, and a hub skirt 124 spaced radially apart from and extending around needle support 122. Hub skirt 124 defines a pair of radially opposed flanges 125 extending therefrom for engagement with threads (not shown) formed in collar 20 of syringe barrel 12, upon engagement of needle hub 120 to distal end 16 of syringe barrel 12. Needle support 122 is configured and dimensioned for insertion into elongate barrel tip 18 of syringe barrel 12 in order to establish fluid communication with the cavity of syringe barrel 12.

While a Luer-type connector is shown and described, it is contemplated that any type of mechanical connector may be used, including and not limited to threads and bayonet-type structures.

As seen in FIGS. 1-8, multi-functional safety shield 130 includes a distal segment 132, a proximal segment 134 connected to distal segment 132, a foot or retention member 136 operatively connected to proximal segment 134, and a nose member 138 operatively connected to distal segment 132.

Distal segment 132 includes a body portion 140 having a distal end 140a and a proximal end 140b. Body portion 140 of distal segment 132 defines a longitudinal channel 142 (see FIG. 7) which extends along the length thereof. Channel 142 is dimensioned and configured to receive a length of syringe needle 110 therein.

Body portion 140 of distal segment 132 has an upper wall 144 and a pair of spaced apart side walls 146a, 146b each of which extends between proximal end 140b and distal end 140a of distal segment 132. Body portion 140 of distal segment 132 further includes a rear wall 148 extending from and between upper wall 144 and side walls 146a, 146b. Rear wall 148 includes a recess or aperture 148a formed therein for selectively receiving a portion of syringe needle 110, when safety shield apparatus 130 is in an extended position, as will be described in greater detail below.

With continued reference to FIGS. 1-8, proximal segment 134 includes a body portion 150 having a distal end 150a and a proximal end 150b. Body portion 150 of proximal segment 134 defines a longitudinal channel 152 (see FIG. 7) which extends along the length thereof. Channel 152 is dimensioned and configured to receive a length of syringe needle 110 therein.

Body portion 150 of proximal segment 134 includes a top surface or wall 154 and a pair of spaced sidewalls 156a, 156b each of which extends between proximal end 150b and distal end 150a of proximal segment 134. Spaced sidewalls 156a, 156b of proximal segment 134 are dimensioned to be receive within or extend around side walls 146a, 146b of distal segment 132 when safety shield apparatus 130 is in a retracted position.

Body portion 150 of proximal segment 134 further includes a front wall 158 extending from and between top wall 154 and side walls 156a, 156b. Front wall 158 includes a recess or aperture 158a formed therein for selectively receiving a portion of syringe needle 110, when safety shield apparatus 130 is in an extended position, as will be described in greater detail below. Aperture 158a of front wall 158 is axially aligned with aperture 148a of rear wall 148 of distal segment 132.

In one embodiment, top surface 154 of body portion 152 of proximal segment 134 includes a thumb engagement member 154a which is ribbed to provide a slip-resistant thumb engaging surface.

With continued reference to FIGS. 1-8, safety shield apparatus 130 includes a hinge member 160 inter-connecting distal segment 132 and proximal segment 134. In particular, hinge member 160 is formed integrally between distal segment 132 and proximal segment 134. Alternately, hinge member 160 can be formed as separate matable components formed in or extending from distal segment 132 and proximal segment 134. As seen in FIGS. 1-8, hinge member 160 can be formed as a thinned transition region and act as a living hinge which is integrally formed between distal segment 132 and proximal segment 134.

As seen in FIGS. 1-8, retention member 136 of safety shield apparatus 130 is monolithically or integrally formed with proximal segment 134 and is hingedly connected to proximal end 150b of proximal segment 134 by a thinned transition region or living hinge 162 (see FIG. 2). Alternately, retention member 136 and proximal segment 134 may be formed separately and pivotally attached to one another with a separate hinge member. Retention member 136 includes a base portion 136a which defines a mounting hole (not shown) for securing safety shield apparatus 130 to a distal end of needle support 122 of needle hub 120. Retention member 136 further includes a latch member 136b integrally formed with and extending from base portion 136a. Latch member 136b is configured and adapted to selectively engage body portion 150 of proximal segment 134 when safety shield apparatus 130 is in a retracted position so as to maintain safety shield apparatus 130 in the retracted position. It is contemplated that latch member 136b may engage body portion 150 of proximal segment 134 in a snap-fit manner.

With continued reference to FIGS. 1-8, nose member 138 of safety shield apparatus 130 is monolithically or integrally formed with distal segment 132 and is hingedly connected to distal end 140a of distal segment 132 by a thinned transition region or living hinge 164. Alternately, nose member 138 and distal segment 132 may be formed separately and pivotally attached to one another with a separate hinge member.

Nose member 138 includes an elongate body portion 170 defining a lumen 172 therein. Lumen 172 of body portion 170 is configured and dimensioned for slidable support on syringe needle 110. Body portion 170 of nose member 138 terminates in a tapered distal end 170a. In use, as will be described in greater detail below, when safety shield apparatus 130 is in a retracted position, distal end 110a of syringe needle 110 extends from distal end 170a of body portion 170 of nose member 138, and when safety shield apparatus 130 is in an extended position, distal end 110a of syringe needle 110 is located proximally of distal end 170a of body portion 170 of nose member 138.

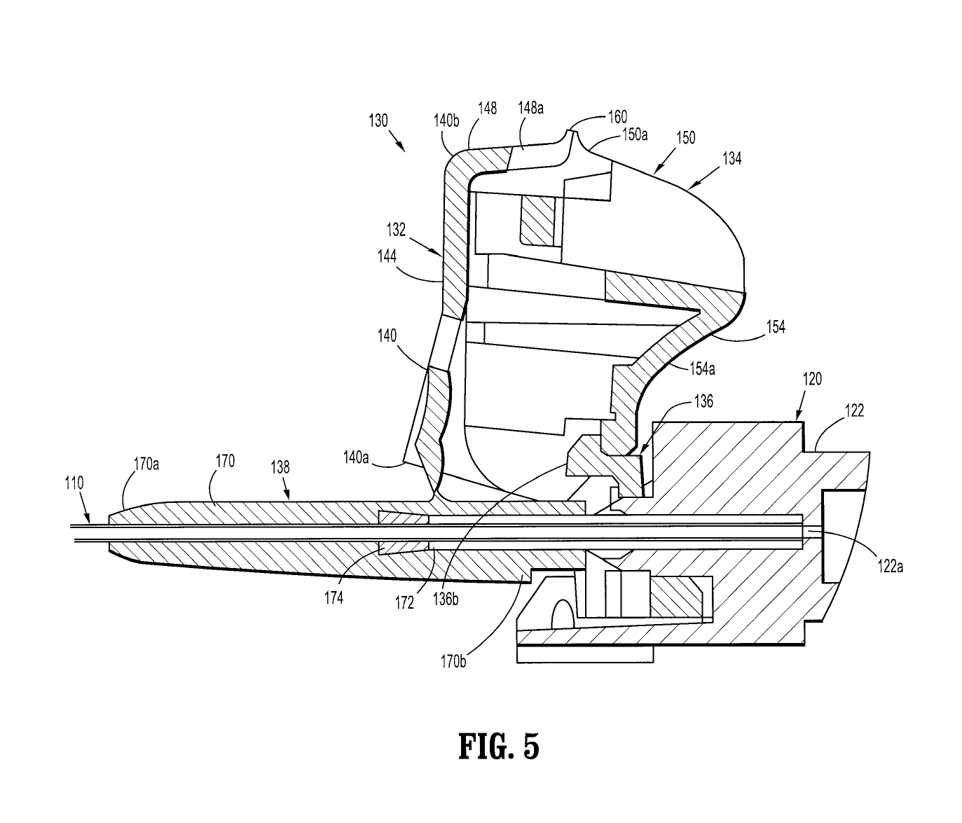

As best seen in FIG. 5, nose member 138 includes a sealing member 174 supported in lumen 172 and surrounding syringe needle 110. Sealing member 174 may be in the form of any suitable compressible/elastomeric material, viscous material or the like capable of permitting relatively free axial movement of nose member 138 relative to syringe needle 110 and capable of establishing a fluid tight seal between nose member 138 and syringe needle 110. A suitable sealing member 174 may include and is not limited to a septum seal, an O-ring, a wiper seal or the like. A wiper seal may include a disk-like body portion defining an opening formed therein, wherein at least a portion of the body portion has a tapered cross-sectional profile extending toward the opening. Sealing member 174 may further include a gel, a putty or a grease-like substance.

A lubricant may be coated onto or impregnated in syringe needle 110, nose member 138 of safety shield apparatus 130 and/or in sealing member 174. A suitable lubricant that may be impregnated into sealing member 174 includes and is not limited to siloxane. Suitable sealing members 174 may be manufactured from ultra-high molecular weight functionalized siloxane polymer dispersed in high density polyethylene, sold under the tradename MB50-314 Masterbatch, available from Dow Corning®, Midland, Mich.

Sealing member 174 may further include a hydrogel or other suitable material capable of swelling upon contact with a liquid, wherein the hydrogel sealing member is relatively dry during extension of safety shield apparatus 130 and swells upon contact with a liquid during use of medical access device 100 while safety shield apparatus 130 is in the extended position. In this manner, hydrogel sealing member allows for relative free axial movement of nose member 138 relative to syringe needle 110 and swells, during use of medical access device 100 while safety shield apparatus 130 is in the extended position, to establish a fluid tight seal between nose member 138 and syringe needle 110.

In one embodiment, sealing member 174 may include a hydrophilic wicking material, a hydrophobic plug or a combination thereof.

In an alternate embodiment, lumen 172 of nose member 138 may have a radially expanding diameter extending in a proximal direction, and syringe needle 110 may have a radially expanding diameter extending in a distal direction. In this manner, as safety shield apparatus 130 is actuated or moved from the retracted position to the extended position, an interference or friction fit or engagement is established between nose member 138 and syringe needle 110.

Referring to FIGS. 1-5, in its retracted position, safety shield apparatus 130 is supported on needle hub 120 such that a longitudinal axis of each of proximal segment 134 and distal segment 132 thereof is substantially perpendicular to a longitudinal axis of syringe needle 110. In the retracted position of safety shield apparatus 130, latch member 136b of retention member 136 is releasably engaged with top surface 154 of body portion 150 of proximal segment 134 to releasably lock safety shield apparatus 130 in its retracted position. Further, when safety shield apparatus 130 is in its retracted position, distal end 110a of syringe needle 110 extends from distal end 170a of body portion 170 of nose member 138.

While safety shield apparatus 130 is in the retracted position, and distal end 110a of syringe needle 110 is exposed, medical access device 100 may be used as any conventional medical needle to withdraw blood from a subject, to access a vial and withdraw medication therefrom, to penetrate a sealed container, or the like.

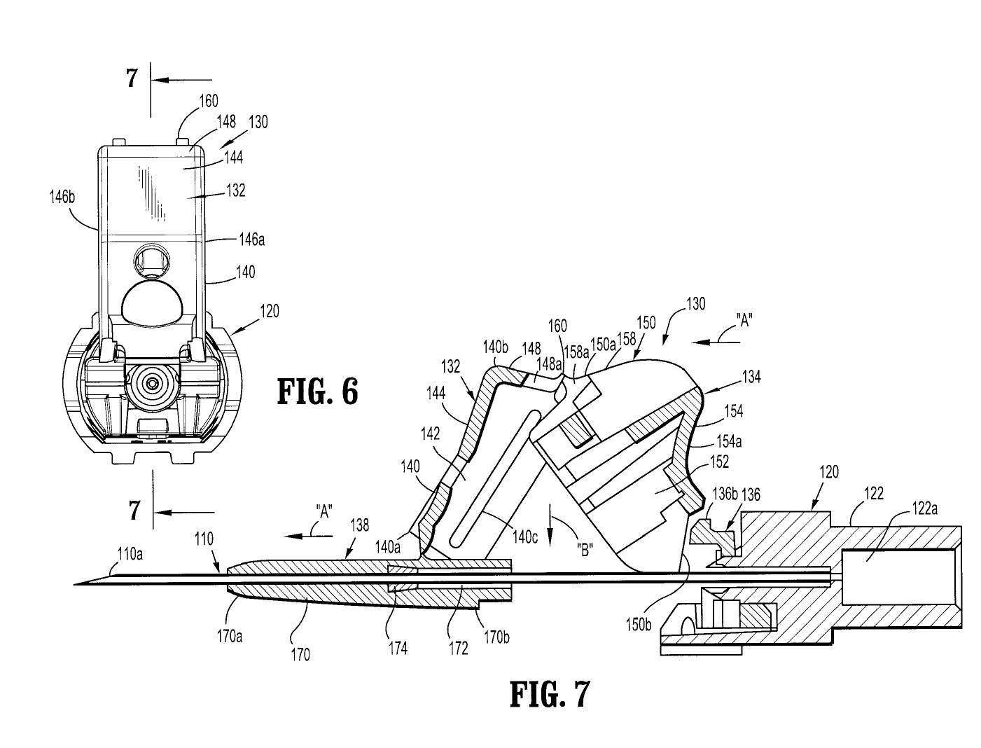

Referring now to FIGS. 6 and 7, safety shield apparatus 130 is moved from its retracted position to its extended position by manually pressing on thumb engaging member 154a of proximal segment 134 in the direction indicated by arrow “A”. As illustrated, by pressing on thumb engaging member 154a, in the direction of arrow “A”, latch member 136b of retention member 136 is disengaged from body portion 150 of proximal segment 134 and nose member 138 is moved axially, in the direction or arrow “A”, along a length of syringe needle 110.

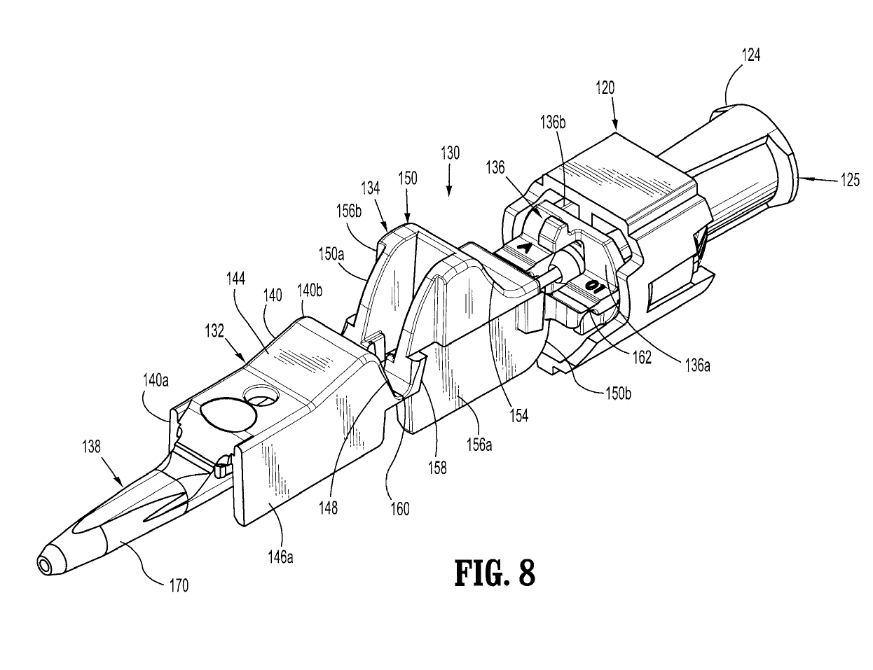

Referring to FIG. 7, as safety shield apparatus 130 is moved in the direction, as indicated by arrow “B”, towards its extended position, hinge member 160 is moved towards syringe needle 110. As distal segment 132 of safety shield apparatus 130 is moved further distally, a distal tip of nose member 138 is moved distally beyond distal end 110a of syringe needle 110. See FIG. 8.

With continued reference to FIG. 7, as distal segment 132 of safety shield apparatus 130 is moved towards syringe needle 110, such that safety shield apparatus 130 is in the fully extended position, engaging members 140c formed in side walls 146a, 146b of body portion 140 of distal segment 132 engage with proximal end 170b of nose member 138.

Engaging members 140c may be in the form of tabs, fingers ribs or the like projecting or extending inwardly of side walls 146a, 146b. In this manner, as safety shield apparatus 130 is moved to the fully extended position, engaging members 140c snap-over or otherwise suitable engage the outer surface of proximal end 170b of nose member 138 or of complementary engaging structure formed on or in the outer surface of nose member 138. Inter-engagement of engaging members 140c with proximal end 170b of nose member 138 helps or functions to maintain or lock safety shield apparatus 130 in the fully extended position.

When safety shield apparatus 130 is in a fully extended position, syringe needle 110 is disposed in aperture 158a of front wall 158 of proximal segment 134 and in aperture 148a of rear wall 148 of distal segment 132.

When safety shield apparatus 130 is in a fully extended position, as seen in FIG. 8, distal end 110a of syringe needle 110 is located proximally of distal end 170a of body portion 170 of nose member 138 or, in other words, fully disposed within nose member 138. In this manner, distal end 110a of syringe needle 110 is shielded and thus a clinician is protected from inadvertent or accidental sticking by distal end 110a of syringe needle 110 once use of syringe needle 110 of medical access device 100 has been completed. Also, when safety shield apparatus 130 is in the fully extended position sealing member 174 is located proximal of distal end 110a of syringe needle 110.

Additionally, when safety shield apparatus 130 is in a fully extended position, medical access device 100 may be used as a blunt tip access cannula or device. As so configured, medical access device 100 may be used to access membranes or septums (e.g., pre-slit septums) of medication vials, intravenous bags, access ports and the like. In particular, the distal end of nose member 138 is introduced into or through a membrane or septum thereby eliminating any damage or coring of the membrane or septum.

In use, once distal end of nose member 138 is penetrated or passed through the membrane or septum of the underlying container, medical access device 100 is used to inject fluid into the underlying container or withdraw fluid from the underlying container. The fluid is communicated into or out of the underlying container through lumen 172 of body portion 170 of nose member 138 and through the lumen of syringe needle 110. The membrane or septum of the underlying container establishes a fluid tight seal around an exterior of body portion 170 of nose member 138, and sealing member 174, as described above, establishes a fluid tight seal between nose member 138 and syringe needle 110.

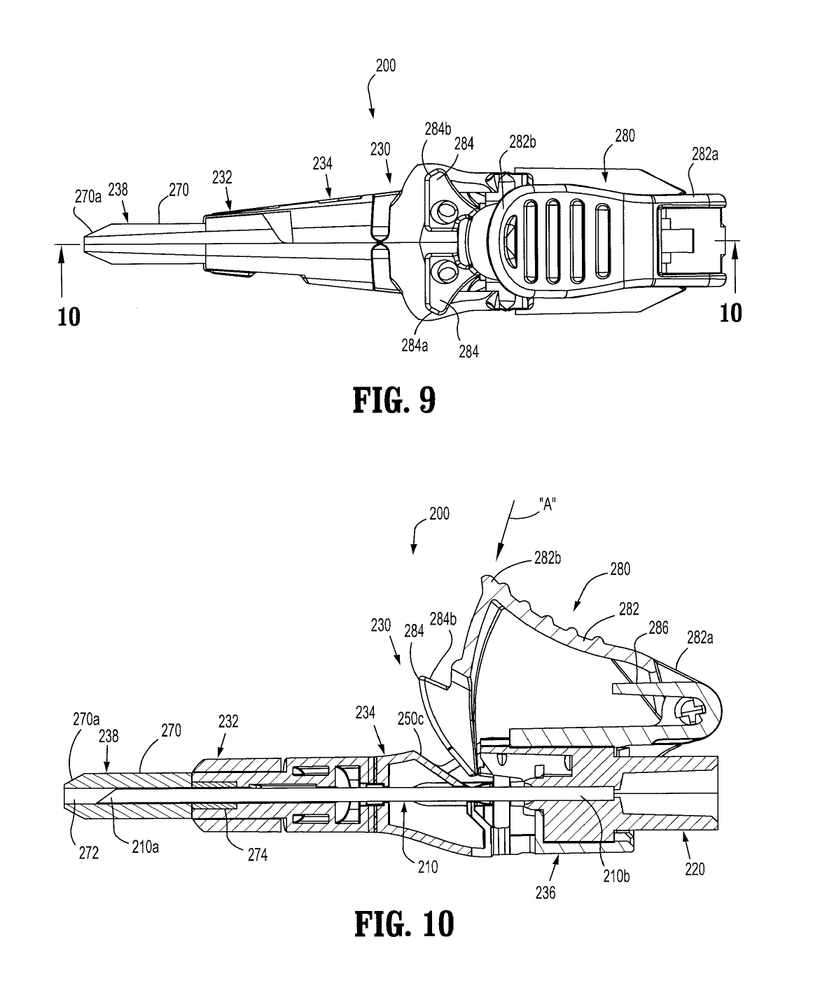

Turning now to FIGS. 9-12, a medical access device according to another embodiment of the present disclosure, is generally shown as 200.

Similar to medical access device 100, medical access device 200 includes a syringe needle 210 supported on a needle hub 220, and a multi-functional safety shield 230 operatively mounted on needle hub 220 and slidably positioned about syringe needle 210. A distal end 210a of syringe needle 210 is tapered to enable tissue penetration and the like, a proximal end 210b of syringe needle 210 is fluidly connected to or supported within needle hub 220.

Needle hub 220 is substantially similar to needle hub 120 and thus will not be discussed in further detail herein. Reference may be made to needle hub 120 for a detailed explanation of the features and elements of needle hub 220.

As seen in FIGS. 9-12, multi-functional safety shield 230 includes a distal segment 232, a proximal segment 234 connected to distal segment 232, a foot or retention member 236 operatively connected to proximal segment 234, and a nose member 238 operatively connected to distal segment 232.

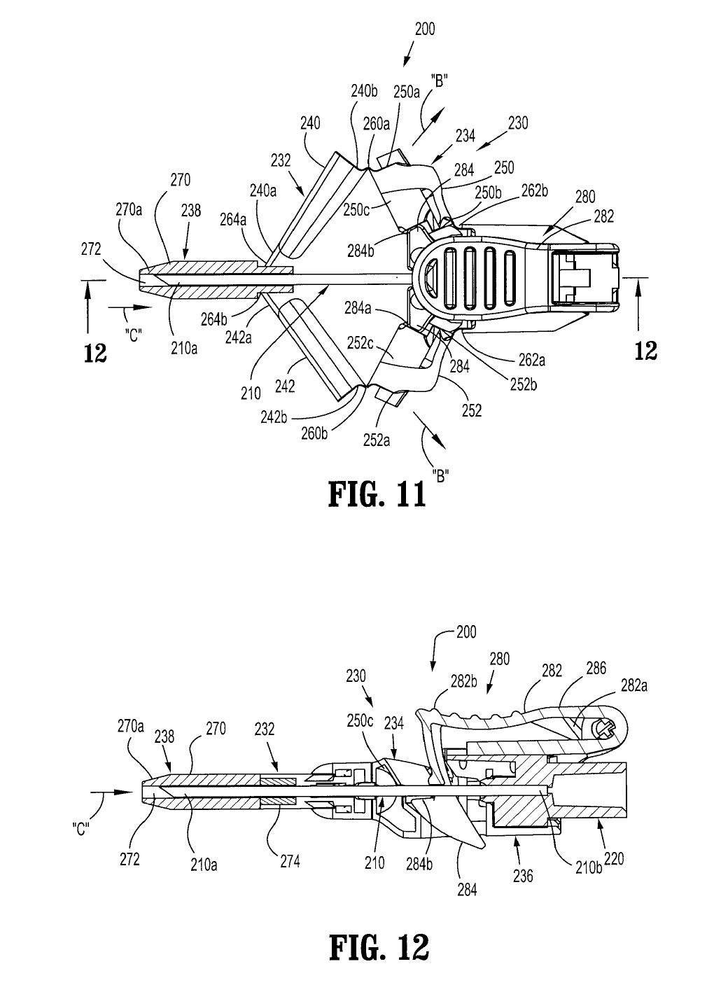

As seen in FIG. 11, distal segment 232 includes a pair of body halves 240, 242 each having a distal end 240a, 242a and a proximal end 240b, 242b. Body halves 240, 242 of distal segment 232 define a longitudinal channel (not shown), which extends along the length thereof. The channel defined by body halves 240, 242 is dimensioned and configured to receive a length of syringe needle 210 therein.

With continued reference to FIG. 11, proximal segment 234 includes a pair of body halves 250, 252 each having a distal end 250a, 252a and a proximal end 250b, 252b. Body halves 250, 252 of proximal segment 234 define a longitudinal channel (not shown), which extends along the length thereof. The channel defined by body halves 250, 252 is dimensioned and configured to receive a length of syringe needle 210 therein.

As seen in FIG. 11, a proximal end of body half 240 of distal segment 232 is hingedly connected to a distal end of body half 250 of proximal segment 234 by a hinge member 260a. Likewise, a proximal end of body half 242 of distal segment 232 is hingedly connected to a distal end of body half 252 of proximal segment 234 by a hinge member 260b. Hinge members 260a, 260b can be formed as separate matable components formed in or extending from respective body halves of distal segment 232 and proximal segment 234, or can be formed as a thinned transition region and act as a living hinge which is integrally formed between respective body halves of distal segment 232 and proximal segment 234.

As seen in FIG. 11, retention member 236 of safety shield apparatus 230 is monolithically or integrally formed with proximal segment 234 and is hingedly connected to body halves 250, 252 of proximal segment 234 by thinned transition regions or living hinges 262a, 262b. Alternately, retention member 236 and body halves 250, 252 of proximal segment 234 may be formed separately and pivotally attached to one another with a separate hinge member.

With continued reference to FIGS. 9-12, nose member 238 of safety shield apparatus 230 is monolithically or integrally formed with distal segment 232 and is hingedly connected to the distal end of each body half 240, 242 of distal segment 232 by a respective thinned transition region or living hinge 264a, 264b. Alternately, nose member 238 and body halves 240, 242 of distal segment 232 may be formed separately and pivotally attached to one another with a separate hinge member.

Nose member 238 includes an elongate body portion 270 defining a lumen 272 therein. Lumen 272 of body portion 270 is configured and dimensioned for slidable support on syringe needle 210. Body portion 270 of nose member 238 terminates in a tapered distal end 270a. In use, as will be described in greater detail below, when safety shield apparatus 230 is in a retracted or primed position, distal end 210a of syringe needle 210 is contained within distal end 270a of body portion 270 of nose member 238, and when safety shield apparatus 230 is in a fully extended position, distal end 210a of syringe needle 210 is further contained within distal end 270a of body portion 270 of nose member 238.

As best seen in FIGS. 10 and 12, nose member 238 includes a sealing member 274 supported in lumen 272 and surrounding syringe needle 210. Sealing member 274 may be substantially similar to sealing member 174 and thus will not be discussed in further detail herein. Reference may be made to sealing member 174 for a detailed explanation of sealing member 274.

As seen in FIGS. 9-12, medical access device 200 further includes a trigger mechanism 280 supported on needle hub 220 or retention member 236, and selectively, operatively associated with body halves 250, 252 of proximal segment 234. Trigger mechanism 280 includes a lever 282 pivotably attached at one 282a end thereof to needle hub 220 or retention member 236, and a second end 282b disposed over or extending over body halves 250, 252 of proximal segment 234.

Trigger mechanism 280 includes a camming member 284 extending from second end 282b of lever 282, in a direction toward body halves 250, 252 of proximal segment 234. Camming member 284 of trigger mechanism 280 is configured and dimensioned to selectively engage camming surfaces 250c, 252c formed in body halves 250, 252 of proximal segment 234. In operation, as will be described in greater detail below, camming member 284 is configured and dimensioned to press against camming surfaces 250c, 252c to force body halves 250, 252 of proximal segment 234 apart from one another whilst not obstructing or interfering with the passage of syringe needle 210.

As seen in FIGS. 10 and 12, trigger mechanism 280 includes a biasing member 286 operatively associated with lever 282 and being configured and adapted to maintain camming member 284 spaced away from camming surfaces 250c, 252c of body halves 250, 252 of proximal segment 234.

Referring to FIGS. 9 and 10, in its extended position, safety shield apparatus 230 is configured such that body halves 240, 242 of distal segment 232 and body halves 250, 252 of proximal segment 234 are axially aligned with one another and are substantially parallel with a longitudinal axis of syringe needle 210. In the extended position, distal end 210a of syringe needle 210 is located proximally of distal end 270a of body portion 270 of nose member 238 or, in other words, fully disposed within nose member 238. Additionally, while in the extended position, camming member 284 of trigger mechanism 280 may be spaced from or may rest against camming surfaces 250c, 252c of body halves 250, 252 of proximal segment 234.

Medical access device 200 is configurable to a primed position, wherein body halves 240, 242 of distal segment 232 and body halves 250, 252 of proximal segment 234 are spaced away from the longitudinal axis and nose member 238 is withdrawn proximally to a position where distal end 210a of syringe needle 210 is still disposed within lumen 272 of body portion 270 of nose member 238. To configure medical access device 200 to the primed position, as depicted in FIGS. 11 and 12, lever 282 of trigger mechanism 280 is pressed downwardly (i.e., towards syringe needle 210), as indicated by arrow “A”, thereby pressing camming member 284 of trigger mechanism 280 against camming surfaces 250c, 252c of body halves 250, 252 of proximal segment 234. As camming member 284 presses against camming surfaces 250c, 252c of body halves 250, 252, body halves 250, 252 of proximal segment 234 are moved apart from one another (i.e., away from syringe needle 210), as indicated by arrows “B”.

Lever 282 of trigger mechanism 280 is pressed until shoulders 284a, 284b move beneath or snap-under camming surfaces 250c, 252c of body halves 250, 252. Additionally, as lever 282 of trigger mechanism 280 is pressed in the direction of arrow “A”, lever 282 presses against and biases or flexes biasing member 286.

In the primed position, medical access device 200 may be used as any conventional medical needle to inject and withdraw blood from a subject, to withdraw medication from a vial, or the like. In particular, while medical access device 200 is in the primed position, as shown in FIGS. 11 and 12, any subsequently applied longitudinal forces acting on nose member 278, in a proximal direction relative to syringe needle 210 (as indicated by arrow “C” of FIGS. 11 and 12), will cause body halves 250, 252 of proximal segment 234 to flex outwardly, in the direction of arrows “B”, and unsheathe distal end 210a of syringe needle 210 for use. Performing the injection and withdrawal (i.e., continuing such longitudinal application of force along the longitudinal axis) causes body halves 250, 252 of proximal segment 234 to flex apart to a substantially maximum position. As such, shoulders 284a, 284b of camming member 284 disengage from beneath or are freed from beneath camming surfaces 250c, 252c of body halves 250, 252. With shoulders 284a, 284b of camming member 284 disengaged, biasing member 286 urges lever 282 and camming member 284 to rise up, as a result of its own resiliency, and out of engagement with camming surfaces 250c, 252c.

When the longitudinal force acting on nose member 238 is removed, distal end 210a of syringe needle 210 retracts into nose member 238, thereby re-sheathing syringe needle 210. Medical access device 200 may include a biasing member (not shown), in the form of a spring or the like, mounted to needle hub 220 and at least one of body halves 250, 252 of proximal segment 234 to facilitate or assist in the re-sheathing of syringe needle 210. In use, when body halves 250, 252 of proximal segment 234 flex outwardly, in the direction of arrows “B”, the biasing member(s) are biased such that when the longitudinal force acting on nose member 238 is removed or reduced below a level of a spring constant of the biasing member(s), the biasing member(s) cause body halves 250, 252 of proximal segment 234 to move towards syringe needle 210. In so doing, nose member 238 is extended over distal end 210a of syringe needle 210.

In this manner, distal end 210a of syringe needle 210 is once again shielded and thus a clinician is protected from inadvertent or accidental sticking by distal end 210a of syringe needle 210 once use of syringe needle 210 of medical access device 200 has been completed. Also, when safety shield apparatus 230 is in the fully extended position sealing member 274 is located proximal of distal end 210a of syringe needle 210.

Additionally, when safety shield apparatus 230 is in a fully extended position, medical access device 200 may be used as a blunt tip access cannula or device. As so configured, medical access device 200 may be used to penetrate membranes or septums of medication vials, intravenous bags, access ports and the like. In particular, the distal end of nose member 238 is introduced into or through a membrane or septum thereby eliminating any damage or coring of the membrane or septum.

In use, once distal end of nose member 238 is penetrated or passed through the membrane or septum of the underlying container, medical access device 200 is used to inject fluid into the underlying container or withdraw fluid from the underlying container. The fluid is communicated into or out of the underlying container through lumen 272 of body portion 270 of nose member 238 and through the lumen of syringe needle 210. The membrane or septum of the underlying container establishes a fluid tight seal around an exterior of body portion 270 of nose member 238, and sealing member 274, as described above, establishes a fluid tight seal between nose member 238 and syringe needle 210. Furthermore, following use of medical access device 200 as a blunt tip access device or more particularly, when safety shield apparatus 230 is in the fully extended position, safety shield apparatus 230 may be returned to the primed position by pressing lever 282 of trigger mechanism 280 towards syringe needle 210. Accordingly, medical access device 200 may subsequently be used as a conventional medical needle to inject medication, to withdraw blood from a subject, to withdraw medication from a vial, or the like.

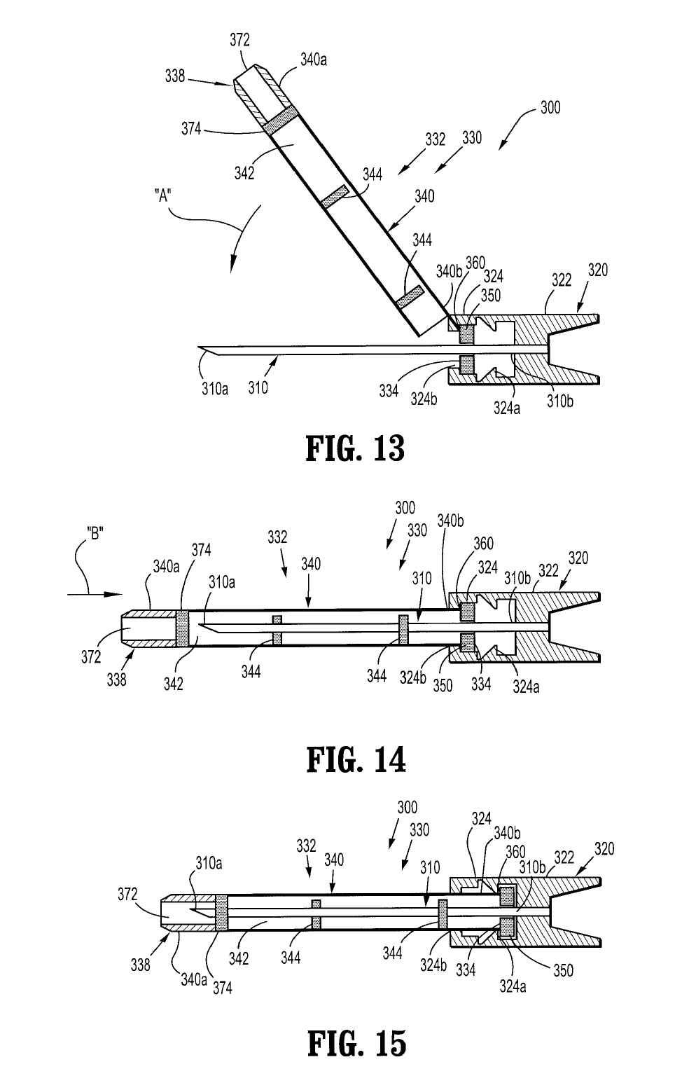

Turning now to FIGS. 13-15, a medical access device according to a further embodiment of the present disclosure is generally designated as 300.

Similar to medical access devices 100 and 200, medical access device 300 includes a syringe needle 310 supported on a needle hub 320, and a multi-functional safety shield 330 operatively mounted on needle hub 320 and slidably positioned about syringe needle 310. A distal end 310a of syringe needle 310 is tapered to enable tissue penetration and the like, a proximal end 310b of syringe needle 310 is fluidly connected to or supported within needle hub 320.

Needle hub 320 is substantially similar to needle hubs 120, 220 and thus will only be discussed in further detail herein to the extent necessary to identify differences in construction and operation. Hub 320 includes a cowl or skirt 324 extending from needle support 322. Skirt 324 includes at least one engagement or locking feature 324a formed therewith, and a distal aperture 324b through which syringe needle 310 extends.

As seen in FIGS. 13-15, multi-functional safety shield 330 includes a distal segment 332 and a proximal segment 334 connected to distal segment 332.

Distal segment 332 includes a body portion 340 having a distal end 340a and a proximal end 340b. Body portion 340 of distal segment 332 defines a longitudinal channel 342 which extends along the length thereof. Channel 342 is dimensioned and configured to receive a length of syringe needle 310 therein. Body portion 340 of distal segment 332 has an upper wall and a pair of spaced apart side walls each of which extends between proximal end 340b and distal end 340a of distal segment 332.

Distal segment 332 further includes at least one retaining member 344 formed in or provided in channel 342 of body portion 340. Retaining members 344 are configured and adapted to selectively engage syringe needle 310 when body portion 340 of distal segment 332 is engaged with or coupled to syringe needle 310. Retaining members 344 are also configured and adapted to allow body portion 340 of distal segment 332 to reciprocate axially along syringe needle 310 when body portion 340 is coupled thereto.

With continued reference to FIGS. 13-15, proximal segment 334 includes a body portion 350 configured and adapted for slidably disposition within skirt 324 of needle hub 320. Proximal segment 334 is hingedly connected to distal segment 332. In one embodiment, safety shield apparatus 330 includes a hinge member 360 formed integrally between distal segment 332 and proximal segment 334. Alternately, hinge member 360 can be formed as separate matable components formed in or extending from distal segment 332 and proximal segment 334. Hinge member 360 can be formed as a thinned transition region and act as a living hinge which is integrally formed between distal segment 332 and proximal segment 334.

Distal end 340a of body portion 340 defines a nose member 338 including a lumen 372 therein. Lumen 372 of nose member 338 is configured and dimensioned for slidable receipt of syringe needle 310. Nose member 338 may terminate in a tapered distal end.

As seen in FIGS. 13-15, nose member 338 includes a sealing member 374 supported in lumen 372. Sealing member 374 may be substantially similar to sealing members 174, 274 and thus will not be discussed in further detail herein. Reference may be made to sealing members 174, 274 for a detailed explanation of sealing member 374. The sealing member may also be and is preferably a septum like structure that is penetrated by the needle upon motion in the “B” direction.

Referring to FIG. 13, in an un-covered or filling configuration, body portion 340 of distal segment 332 of safety shield apparatus 330 is spaced apart from or un-connected to syringe needle 310. While in the un-covered position, proximal segment 334 is located at a distal-most end of skirt 324 and distal of locking feature 324a of needle hub 320.

While in the un-covered configuration, distal end 310a of syringe needle 310 is exposed and medical access device 300 may be used as any conventional medical needle to withdraw blood from a subject, to withdraw medication from a vial, or the like.

Following use of medical access device 300 in the un-covered or filling configuration, as seen in FIGS. 13 and 14, medical access device 300 is configured to a first covered or sliding configuration by rotating distal segment 332 towards syringe needle 310, as indicated by arrow “A” of FIG. 13, until syringe needle 310 is engaged by retaining members 344 and fully disposed within channel 342 defined by body portion 340 of distal segment 332. In the first covered configuration, as seen in FIG. 14, sealing member 374 is located distal of distal end 310a of syringe needle 310 and proximal segment 334 of safety shield apparatus 330 is located distal of locking feature 324a of skirt 324.

With safety shield apparatus 330 in the first covered configuration, medical access device 300 is configured to a second covered or ready-to-use configuration by sliding distal segment 332 and proximal segment 334 proximally relative to needle hub 320, as indicated by arrow “B” of FIG. 14, until proximal segment 334 of safety shield apparatus 330 operatively engages or otherwise snaps-over locking feature 324a of skirt 324. Simultaneously therewith, sealing member 374 is moved proximally relative to syringe needle 310 until distal end 310a of syringe needle 310 penetrates through sealing member 374 and is fully retained within lumen 372 of nose member 338. Accordingly, when safety shield apparatus 330 is in the second covered configuration sealing member 374 is located proximal of distal end 310a of syringe needle 310.

Additionally, when safety shield apparatus 330 is in the second covered configuration, as seen in FIG. 15, medical access device 300 may be used as a blunt tip access cannula or device. As so configured, medical access device 300 may be used to penetrate membranes or septums of medication vials, intravenous bags, access ports and the like. In particular, the distal end of nose member 338 is introduced into or through a membrane or septum thereby eliminating any damage or coring of the membrane or septum.

In use, once distal end of nose member 338 is penetrated or passed through the membrane or septum of the underlying container, medical access device 300 is used to inject fluid into the underlying container or withdraw fluid from the underlying container. The fluid is communicated into or out of the underlying container through lumen 372 of nose member 338 and through the lumen of syringe needle 310. The membrane or septum of the underlying container establishes a fluid tight seal around an exterior of nose member 338, and sealing member 374, as described above, establishes a fluid tight seal around syringe needle 310.

While medical access device 300 is in the first and second covered configurations, distal end 310a of syringe needle 310 is shielded and thus a clinician is protected from inadvertent or accidental sticking by distal end 310a of syringe needle 310 once use of syringe needle 310 of medical access device 300 has been completed.

It will be understood that various modifications may be made to the embodiments disclosed herein. Therefore, the above description should not be construed as limiting, but merely as exemplifications of preferred embodiments. Those skilled in the art will envision other modifications within the scope and spirit of the claims appended hereto.

What is claimed is:

1. A medical access device, comprising:
a syringe needle having a proximal end and a distal end configured for percutaneous use; and
a safety shield supportable on the syringe needle, the safety shield including:
a pair of spaced legs each having a distal segment and a proximal segment, each of the proximal segments having a proximal end fixed with respect to the syringe needle and a distal end hingedly attached to the distal segment at a hinge member, each proximal segment including a camming surface;
a nose member connected to a distal end of each distal segment of the pair of spaced legs, wherein the nose member is translatably disposed on the syringe needle, wherein the nose member defines a blunt tip; and
a trigger having a camming member and being movable to move the camming member into engagement with the camming surfaces of the proximal segments to effect movement of the legs between:
a first position in which the nose member and the distal ends of the distal segments shield at least a distal end of the syringe needle; and
a second position in which the distal end of the syringe needle is shielded by the nose member and capable of being at least partially exposed from a distal end of the nose member when the nose member is moved in a proximal direction relative to the syringe needle.

2. The medical access device according to claim 1, wherein in the first position, the distal and proximal segments are substantially linearly aligned with the hinge member of each leg positioned adjacent the needle such that when a force, acting in a substantially linear proximal direction, is applied to a distal end of the nose member of the safety shield, the legs are retained in the first position.

3. The medical access device according to claim 1, wherein the camming member of the trigger and the camming surfaces of the proximal segments are positioned and configured such that movement of the camming member of the trigger into engagement with the camming surfaces of the proximal segments splays the hinge member of each of the legs outwardly to retract the nose member in a proximal direction relative to the syringe needle.

4. The medical access device according to claim 1, wherein each of the legs defines a channel dimensioned to receive the syringe needle.

5. The medical access device according to claim 1, further comprising a needle hub configured for selective connection with a complementary feature of a fluid source, wherein the trigger is pivotally secured to the needle hub.

6. The medical access device according to claim 5, wherein the proximal end of the syringe needle is pivotably connected to the needle hub, and is axially movable with respect to the needle hub.

7. The medical access device according to claim 6, wherein the needle hub includes a locking feature configured to maintain the syringe needle in a retracted position.

8. The medical access device according to claim 1, further comprising a biasing member operatively associated with a lever of the trigger and configured to urge the legs to the first position.

9. The medical access device according to claim 1, wherein the hinge member of each of the legs is constructed as a separate matable component formed in or extending from the legs.

10. The medical access device according to claim 1, wherein the hinge member of each of the legs is constructed as a thinned transition region integrally formed between the legs.

11. The medical access device according to claim 1, wherein the safety shield includes at least one retaining member for inhibiting uncovering of the syringe needle when a body portion of the syringe needle is in the covered position.

12. The medical access device according to claim 11, wherein the at least one retaining member and the legs are formed separately, and pivotally attach to one another with a separate hinge member.

13. The medical access device according to claim 1, wherein the nose member includes a sealing member supported within a lumen of the nose member and surrounding the syringe needle.

14. The medical access device according to claim 13, wherein the sealing member is an elastomeric material capable of permitting relatively free axial movement of the nose member relative to the syringe needle and capable of establishing a fluid tight seal between the nose member and the syringe needle.

15. The medical access device according to claim 13, wherein the sealing member is at least one of a septum seal, an O-ring, and a wiper seal.

16. The medical access device according to claim 13, wherein the sealing member includes a hydrogel capable of swelling upon contact with a liquid, wherein the hydrogel sealing member is relatively dry during extension of the safety shield and swells upon contact with the liquid during use of medical access device, while the safety shield is in the extended position.

17. The medical access device according to claim 1, wherein a lubricant is coated onto or impregnated in the syringe needle.

18. The medical access device according to claim 1, wherein the trigger further includes a lever pivotable attached to a first end of a needle hub or retaining member and a second end disposed over or extending over the legs.

19. The medical access device according to claim 1, wherein the safety shield includes a body portion axially movable with respect to the syringe needle.

20. The medical access device according to claim 19, wherein when the body portion is in a covered position, the body portion is axially displaced relative to the syringe needle by an amount sufficient for a distal end of the syringe needle to extend completely through a sealing member.

21. The medical access device according to claim 20, wherein the distal end of the nose member has a length sufficient to cover the distal end of the syringe needle when the body portion has been moved to a position where the distal end of the syringe needle extends distally beyond the sealing member.

22. A medical access device, comprising:
a syringe needle having a proximal end and a distal end, the syringe needle slidably extending through at least a body portion of a nose member defining a blunt tip and defining a longitudinal axis therethrough;
a pair of spaced legs each having a distal segment and a proximal segment, the distal segments cooperating with the proximal segments via hinge members, the legs adapted to act as a safety shield pivotally connected to the syringe needle via at least one retention member; and
a trigger mechanism supported on a needle hub or the retention member, the trigger mechanism having a lever and a camming member, the trigger mechanism configured to move the camming member into engagement with camming surfaces of the proximal segments of the legs to effect movement of the legs between a first position and a second position, the movement of the legs being perpendicular to the longitudinal axis defined by the syringe needle.

23. The medical access device according to claim 22, wherein in the first position, the nose member and distal ends of the distal segments shield at least a distal end of the syringe needle.

24. The medical access device according to claim 22, wherein in the second position, a distal end of the syringe needle is shielded by the nose member and is capable of being at least partially exposed from a distal end of the nose member, when the nose member is moved in a proximal direction relative to the syringe needle.

25. The medical access device according to claim 22, further comprising a biasing member operatively associated with the lever of the trigger mechanism and configured to urge the legs to the first position.

26. The medical access device according to claim 22, wherein the nose member includes a sealing member supported within a lumen of the nose member and surrounding the syringe needle.

27. The medical access device according to claim 26, wherein the sealing member is an elastomeric material capable of permitting relatively free axial movement of the nose member relative to the syringe needle and capable of establishing a fluid tight seal between the nose member and the syringe needle.
